(19) United States
(12) Patent Application Publication
(10) Pub. No.: US 2005/0129752 A1
(43) Pub. Date: Jun. 16, 2005

(54) Use and manufacturing process for liposomal doxorubicin pharmaceutical composition

(75) Inventors: Te-Jung Chen, Taipei (TW); Sze-Yuan Yang, Taipei (TW); Chia-Ning Liu, Taipei (TW); Chun-Ying Huang, Taipei (TW); Jung-Chin Lin, Taipei (TW)

Correspondence Address: Kent H. Cheng, Esq.;Cohen, Pontani, Lieberman & Pavane, Suite 1210, 551 Fifth Avenue, New York, NY, 10176, US

(73) Assignee: TTY Biopharm limited company

(21) Appl. No.: 10/957,027

(22) Filed: Oct. 1, 2004

Related U.S. Application Data
(63) Continuation-in-part of application No. 10/734,272, filed on Dec. 15, 2003.

(51) Int. Cl. A61K 31/704; A61K 9/127

(52) U.S. Cl. 424/450; 514/34

(57) ABSTRACT

The present invention provides a method of treating mammals having pancreatic cancer by administering a liposomal doxorubicin pharmaceutical composition, and a process of manufacturing the composition.

### CROSS REFERENCE TO RELATED APPLICATIONS

This is a continuation in part of U.S. patent application Ser. No. 10/734,272, titled “PROCESS FOR PRODUCING LIPOSOME SUSPENSION AND PRODUCT CONTAINING LIPOSOME SUSPENSION PRODUCED”, filed Dec. 15, 2003.

### BACKGROUND OF THE INVENTION

1. Field of the Invention

This invention relates to a process for manufacturing a liposomal doxorubicin pharmaceutical composition, and its use for treating mammals having pancreatic cancer.

2. Description of the Related Art

In 2003, approximately 30,000 patients died of pancreatic cancer, and around 30,700 new cases are diagnosed in the United States, making pancreatic cancer one of the ten most common causes of cancer-related mortality. Surgery, radiation therapy, and chemotherapy are treatment options that can extend survival time and/or relieve symptoms in many patients, but seldom produce a cure. (Cancer Facts and Figures, American Cancer Society 2003).

Liposomal drugs are relatively successful in reaching primary tumors and their metastases after intravenous injection to animals and humans, and show greater therapeutic efficacy and slighter side effects than non-liposomal drugs. One of the most developed liposomal drugs is liposomal doxorubicin. The most common brand name for this drug in Taiwan is Lipo-Dox®, exclusively manufactured and sold by TTY Biopharm Company Ltd. and a similar product is known as Doxil® in the US. Liposomal doxorubicin is commonly used to treat certain cancers, including breast and ovarian cancers, and a type of sarcoma called AIDS-Related Kaposi's sarcoma.

Preparing a liposomal drug is difficult for the pharmaceutical industry. Many methods have been disclosed for producing liposome and/or liposomal drugs; for example, Professor Szoka's invention.

Francis C. Szoka, Jr. in the U.S. Pat. Nos. 5,077,057, 5,277,914, and 5,549,910 disclosed a method for preparing a lipid suspension of defined particle size encapsulating a useful compound with poor water, alcohol or halogenated hydrocarbon solubility. The poorly-soluble compound and a sufficient amount of a suitable lipid are dissolved in an aprotic solvent such as DMSO, optionally containing a solubilizing amount of lower alcohol (for example, ethanol), and then the mixture is extruded or injected into a stirred aqueous solution. The resulting liposomal suspension may be dialyzed or otherwise concentrated, if desired. The extrusion may be performed using a syringe, a perforated plate or tube or any other appropriate device with aperture sizes of about 0.05 mm to about 5 mm. In example 2 of the three U.S. patents, doxorubicin is dissolved in DMSO and added to an ethanol solution containing egg phosphatidylglycerol(EPG): egg phosphatidylcholine(EPC):cholesterol(Chol)(7:3:6). Liposomes are formed by injecting the lipid-doxorubicin mixture into an aqueous phase consisting of 140 mM NaCl-10 mM Tris-HCl, pH4.0, at 30 [symbol] . The liposome suspension is dialyzed and the liposome-encapsulated doxorubicin is separated from the non-encapsulated material by column chromatography. The resulting liposome particle diameter is 277 nm, and 41.2% of the doxorubicin is encapsulated in the liposome particles.

However, the previous process could not eliminate certain problems, such as toxic organic solvents, maintenance of aseptic conditions, and uniformity and quality of liposomes; thus, they are unsuitable for a large-scale production.

A method for treating pancreatic cancer with liposomal doxorubicin has previously been disclosed; for example, “A Phase II Study evaluating the tolerability and efficacy of CAELYX (liposomal doxorubicin, Doxil) in the Treatment of Unresectable Pancreatic Carcinoma” (Ann Oncol. 2001 October; 12(10):1399-402). However, this study concluded that no objective responses were seen with CAELYX® (liposomal doxorubicin, Doxil®). However, the regimen that was used might not have been effective in treating the disease.

The present invention, in addition to providing a novel process for preparing liposomal doxorubicin that is well-suited to industrial production, provides a method for treating pancreatic cancer with the liposomal doxorubicin. In order to assess efficacy, the tumor size and survival time for rats bearing pancreatic tumor with and without liposomal doxorubicin administration was studied.

All literature and patents mentioned above, as well as the literature cited thereafter, are incorporated herein by reference in their entirety.

### SUMMARY OF THE INVENTION

Accordingly, it is a goal of the present invention to manufacture a liposomal doxorubicin pharmaceutical composition and to effectively use it to treat pancreatic cancer.

In one aspect, the invention provides a method of treating mammals having pancreatic cancer, comprising administering a therapeutically effective amount of the liposomal doxorubicin pharmaceutical composition.

In another aspect, the invention provides a process for producing the liposomal doxorubicin pharmaceutical composition.

### BRIEF DESCRIPTION OF THE DRAWINGS

In the drawings:

FIG. 1 shows the effect of Lipo-Dox® on rats bearing AR42J pancreatic tumor relative to tumor volume,

and FIG. 2 shows the effect of Lipo-Dox® on rats bearing AR42J pancreatic tumor relative to survival time.

### DETAILED DESCRIPTION OF THE PRESENTLY PREFERRED EMBODIMENTS

The present invention provides a process for producing lipsomal doxorubicin that comprises: (a) providing a pre-mixture comprising 40-70% distearoyl phosphotidylcholine (DSPC), 10-30% cholesterol, and 15-30% methoxy-polyethyleneglycol-distearoyl phosphatidylethanolamine(mPEG-DSPE) to an alcohol solvent in a ratio of around 5:1, at around 55-65° C., (b) mixing the pre-mixture with an aqueous 0.2-0.8N ammonium sulfate solution in a ratio of around 1:10 (v/v) to form a mixture, (c) subjecting the mixture to a pore-extrusion treatment with apertures of 0.05-0.45 μm to form a pre-liposome suspension, at 50-70° C., preferably at 60° C.; (d) dialyzing the pre-liposome suspension with a 5% to 15% aqueous sucrose solution at room temperature, such that a liposome suspension is obtained; and (e) mixing doxorubicin and the liposome suspension obtained from step (d) at 55-65° C., preferably 60° C., in sucrose solution.

The invention, when compared with conventional methods, can be performed at low pressure (about 40 to 140 psi) with higher yields (about 2 to 10 L/minute), which is better than at about 100 to 200 psi and yields of 1 to 5 L/minute for conventional methods.

Rats with pancreatic cancer were treated with Lipo-Dox® by intravenous (I.V.) injection at a dosage of 10 mg/kg. The results are shown in the following description.

Changes of body weight, tumor size, and survival time after administration of normal saline or Lipo-Dox® are shown in Table. 1, and Table. 2.

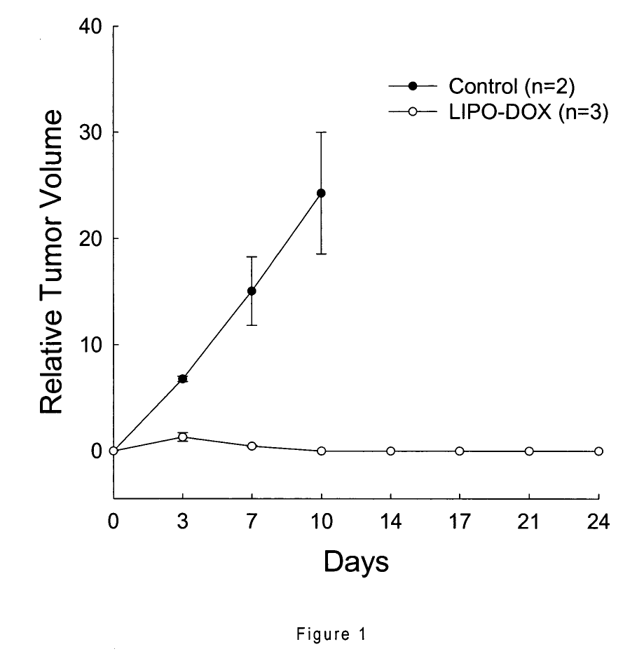

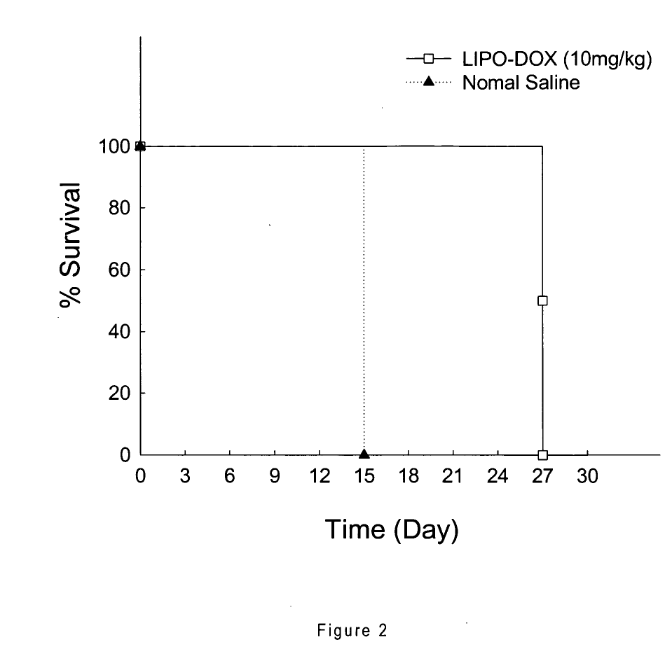

FIG. 1 shows the change in relative tumor volume over time after administration of normal saline (control) or Lipo-Dox® at a dosage of 10 mg/kg. Treatment was started on day 0, 13 days after tumor implantation into rats. Rats were treated on Days 0, 3, 7, and 10 with normal saline or Lipo-Dox® (10 mg/kg). Tumors were measured on the indicated days. The relative tumor volume is expressed as the Vₜ/V₀ index, where Vₜ is the tumor volume on a given day of measurement and V₀ is the volume of the same tumor at the start of the treatment.

FIG. 2 shows the survival time of rats after administration of normal saline (control) or Lipo-Dox® at a dosage of 10 mg/kg. Treatment was started on day 0, 13 days after tumor implantation into rats. Rats were treated on Days 0, 3, 7, and 10 with normal saline or Lipo-Dox® (10 mg/kg).

TABLE 1
The changes of body weight after treating with or without Lipo-Dox ®.

| | Body Weight (g) | | | | | | | |
|---|---|---|---|---|---|---|---|---|
| | Before | After injection | | | | | | |
| No | injection | 3 Days | 7 Days | 10 Days | 14 Days | 17 days | 21 Days | 24 Days |
| Normal | | | | | | | | |
| saline group | | | | | | | | |
| 303 | 180 | 180 | 220 | 230 | Dead | Dead | Dead | Dead |
| 308 | 180 | 180 | 240 | 225 | 240 | Dead | Dead | Dead |
| LIPODOX | | | | | | | | |
| 304 | 170 | 180 | 170 | 120 | 110 | 110 | 110 | 110 |
| 305 | 180 | 190 | 180 | 120 | 110 | 110 | 110 | 110 |
| 311 | 160 | 170 | 170 | 120 | 110 | 110 | 110 | 110 |

TABLE 2
The changes of tumor volume and survival time after treating with or without Lipo-Dox ®.

| | | Tumor Volume | | | | | |
|---|---|---|---|---|---|---|---|
| | | (mm³) | | | | | |
| | Before | After injection | | | Decrease or | | Survival |
| No | injection | 3 Days | 7 Days | 10 Days | Increase % | Response | time (d) |
| Normal | | | | | | | |
| saline group | | | | | | | |
| 303 | 397.55 | 2771.72 | 6885.52 | 11258.26 | In 391.92% | Poor | 13 |
| 308 | 595.73 | 3934.75 | 7601.64 | 12050.18 | In 291.11% | Poor | 15 |
| LIPODOX | | | | | | | |
| 304 | 774.04 | 1195.91 | 318.21 | 0 | De 100% | Good | 26 |
| 305 | 785.7 | 671.62 | 338.2 | 0 | De 100% | Good | 27 |
| 311 | 727.59 | 1139.37 | 397.89 | 0 | De 100% | Good | 27 |

Lipo-Dox® was highly efficient against subcutaneously (s.c.) implanted AR43J pancreatic tumor, when injected intravenously on day 0, 3, 7 and 10. All of the animals were cured at the dose of 10 mg/kg, and treated mice exhibited increased survival rates (see FIG. 1 and FIG. 2). After administering saline via intravenous injection four times, the rats' body weight increased steadily; however, rats treated with Lipo-Dox® exhibited decrease in body weight of up to 33.33 percent. It was concluded that the administration of Lipo-Dox® is an effective treatment for pancreatic cancer.

### EXAMPLES

The following examples illustrate methods of preparing, characterizing, and using the composition of the present invention. The examples are not intended to limit the scope of the invention.

### Example 1

### Preparation of liposomal doxorubicin pharmaceutical composition, Lipo-Dox®

16.8 g of PEG-2000-DSPE (Genzyine Co., America), 27.4 g of cholesterol (NOF Co., Japan) and 38.2 g of DSPC(NOF Co., Japan) were added to 600 ml of ethanol in a glass container. The mixture was mixed well at 60° C. While continuously stirring the mixture and maintaining the mixture at 60° C., 4 L of the aqueous ammonium sulfate solution was added to the mixture. At this temperature, the ethanol almost evaporated. Then the mixture was subjected to a pore-extrusion treatment using a 1.5L of filter (Advantec Toyo Kaisha, Ltd., Japan). The pore-extrusion treatment comprised:
(1) filtering the mixture 10 times using a first filtration membrane (142 mm, 0.1 μm); and
(2) filtering the mixture 10 times more using a second filtration membrane (142 mm, 0.05 μm).
   The extrusion pressure was kept at 3 to 10 kg/cm², and the flow rate was about 2 to 10 L/min. 4500 mL of filtration solution was collected and then dialyzed with 30 L 9%(w/w) sucrose solution that was prepared in a 30 KD hollow fiber (A/G Technology, UFP-30-C-6A, 30,000 NM, 4800 cm²). The remaining ethanol was all removed by dialysis. The volume of the collected solution was about 3000 mL, and the collected solution was a liposome suspension that did not contain ethanol.

The 3000 mL of liposome suspension produced was added to a glass container containing 8000 mg doxorubicin HCl (red powder), then 200 mL histidine-sucrose solution previously prepared was continuously added. The mixture was put in a 600C water bath and stirred for 30 minutes, then cooled to about 350° C., diluted with 9% sucrose solution to 4 L and mixed well.

The product was packaged in sterile glass vials to be used as an injectable preparation containing 2.0 mg doxorubicin HCl/mL.

### Example 2

Preparation of AR42J Cell Suspension. AR42J cell line (ATCC, CRL 1492) derived form and azaserine—induced rat pancreatic tumor, was purchased from ATCC. AR42J cells were cultured in Ham's F-12K (Gibco BRL) medium with 20% fetal bovine albumin, 2 mM L-glutamine and 1.5 g/L sodium bicarbonate at 37 under humidified conditions with 95% O₂ and 5% CO₂. Cells were routinely plated at 1×10⁶ onto the T-flask. Cells were harvested by brief incubation with trypsin-EDTA (Gibco BRL) and the cell suspension was centrifuged at 1000 rpm for 10 minutes and adjusted to 1×10⁷ cells/ml. Trypan blue was used to evaluate the viability of the AR42J pancreatic tumor cells.

### Example 3

### Implantation of AR42J Pancreatic Cancer Cell

Five Lewis rats (supplied by National Science Council, Taiwan), each weighting about approximately 160 g-180 g, and all about 3 weeks old, were used for the experiment. Food and water were given ad libitum. The animal care and use procedures were in accordance with the Guide for the Care and Use of Laboratory Animals from the Institute of Nuclear Energy Research. One million AR42J cells were suspended in 0.1 ml medium and were subcutaneously injected into the right limb of the rats. Two weeks after implantation, tumorvolume was estimated by the formula length (mm)×width2 (mm²)/2. The relative tumor volume was expressed as Vₜ/V₀ index, where Vₜ is the tumor volume on the given day of measurement and V₀ is the volume of the same tumor at the start of treatment.

### Example 4

### Administration of Lipo-Dox® Therapeutic Effect

The rats bearing with AR42J pancreatic tumor were divided into two groups, where there were two rats in the control group and the other three are in Lipo-Dox® group. The rats were either given normal saline or Lipo-Dox® (from 5-15 mg/kg, preferably 10 mg/kg), twice a week (on Wednesday and Saturday) for four times by I.V. injection. Careful observations, such as tumor volume, body weight, activity level, and hair loss were required immediately after dosing, and monitored till there were no more rats surviving.

While the present invention has been described in connection with what is considered the most practical embodiments, it is understood that this invention is not limited to the disclosed embodiments but is intended to cover various modifications that are included within the spirit and scope of the broadest interpretation of the present invention.

1. A method of treating mammals having pancreatic cancer, comprising administering a therapeutically effective amount of liposomal doxorubicin pharmaceutical composition to the mammals in a range of from 5 to 15 mg/kg of body weight twice a week.

2. The method of treating pancreatic cancer of claim 1, wherein the therapeutically effective amount of liposomal doxorubicin pharmaceutical composition is 10 mg/kg of body weight.

3. A process for producing liposomal doxorubicin comprising: (a) combining with an alcohol solvent with a pre-mixture comprising compounds of 40-70% distearoyl phosphotidylcholine (DSPC), 10-30% cholesterol, and 15-30% methoxy-polyethyleneglycol-distearoyl phosphatidylethanolamine (mPEG-DSPE) to form a pre-mixture/alcohol solution, wherein a ratio between the compounds and the alcohol solvent is about 1:5 (w/v); (b) mixing the pre-mixture/alcohol solution with an aqueous 0.2-0.8N ammonium sulfate solution at a ratio of about 1:2-10 (v/v) to form a mixture; (c) subjecting the mixture obtained in step (b) to a pore-extrusion treatment with apertures of 0.05-0.45 μm to form a pre-liposome suspension; (d) dialyzing the pre-liposome suspension with a 5% to 15% sucrose aqueous solution at room temperature, such that a liposome suspension containing suspended liposome particle is obtained; and (e) mixing doxorubicin and the liposome suspension obtained from step (d) in sucrose solution.

4. A method of treating mammals having pancreatic cancer, comprising administering a therapeutically effective amount of liposomal doxorubicin pharmaceutical composition to the mammals in a range of from 5 to 15 mg/kg of body weight twice a week, wherein the liposomal doxorubicin is produced by the process of claim 3.

5. The method of claim 4, wherein the therapeutically effective amount of liposomal doxorubicin pharmaceutical composition is 10 mg/kg of body weight.
